(19) United States
(12) Patent Application Publication
(10) Pub. No.: US 2024/0335138 A1
(43) Pub. Date: Oct. 10, 2024

(54) IOT-BASED PODIATRIC ACTIVITY TRACKING AND RECOMMENDATION SYSTEM

(71) Applicant: University of Florida Research Foundation, Incorporated, Gainesville, FL (US)

(72) Inventors: Swarup BHUNIA, Gainesville, FL (US); Reiner DIZON-PARADIS, Gainesville, FL (US); R. James TOUSSAINT, Gainesville, FL (US)

(21) Appl. No.: 18/621,216

(22) Filed: Mar. 29, 2024

Related U.S. Application Data
(60) Provisional application No. 63/494,926, filed on Apr. 7, 2023.

(51) Int. Cl. A61B 5/103; A61B 5/00; A61B 90/00; G16H 15/00; G16H 20/30

(52) U.S. Cl. CPC A61B 5/1038; A61B 5/0024; A61B 5/6807; A61B 5/6843; A61B 5/7275; A61B 5/7405; A61B 5/743; A61B 5/7455; A61B 90/06; G16H 15/00; G16H 20/30; A61B 2090/065; A61B 2560/0462; A61B 2562/0247; A61B 2562/0271; A61B 2562/029; A61B 2562/166

(57) ABSTRACT

Embodiments of the present disclosure are directed to a portable podiatric activity tracking system for monitoring the neuromuscular gait, stance, and/or performance related to the feet of an end user. Embodiments are configured to receive, by a podiatric data central (PODAC) module, podiatry data associated with a respective foot of the end user. The podiatry data is generated at least in part by podiatry-related sensors associated with respective movable podiatry trackers (MOPTs). Embodiments can also generate, based at least in part on the podiatry data, summary data related to foot activity related to the end user. Embodiments can transmit the summary data to an end device associated with the end user. A mobile software application associated with the end device can generate active feedback based at least in part on the summary data, where the active feedback is configured to encourage or discourage the foot activity related to the end user.

### CROSS REFERENCE TO RELATED APPLICATIONS

This application claims priority to U.S. Application No. 63/494,926 filed Apr. 7, 2023, the contents of which are incorporated herein in their entireties by reference.

### TECHNOLOGICAL FIELD

The present disclosure relates to a portable podiatric activity tracking system, and more particularly, to an apparatus, system, and computer-implemented method for monitoring the neuromuscular gait, stance, and performance related to the feet of an end user and providing gait correction recommendations to the end user while also providing real time feedback.

### BACKGROUND

Gait disorders are frequently seen in the context of neuromuscular disorders. The most common neurologic causes include Parkinson's disease, frontal gait disorders, cerebral ataxia, and spasticity due to stroke, multiple sclerosis and/or spinal cord injury. For a number of these gait disorders, the first line of treatment is non-surgical. Treatment modalities include medications to decrease muscle tonicity, physical therapy, and bracing. While bracing techniques have been shown to be effective, there are numerous inherent risks posed to the end user utilizing the bracing techniques. For example, modern braces are designed to fit more intimately than traditional braces of the past. Although the use of modern braces has led to improved efficacy, there has been a documented increase in the incidence of lower extremity skin issues related to structural or environmental stresses related to the modern braces.

Furthermore, the CDC estimates that 37.3 million people in the United States are diabetic and nearly 50% of diabetics suffer from some component of peripheral neuropathy (e.g., damaged nerves which impair sensation in the limbs). Diabetic foot ulcers (DFU) are a serious complication of peripheral neuropathy that results in significant morbidity and mortality. Mortality rates associated with the development of a DFU are estimated to be 5% in the first 12 months, and 5-year mortality rates have been estimated at 42%. In addition, patients with DFUs were also found to have a 2.5× increased risk of death compared with their diabetic counterparts without foot wounds. Considering these findings, the medical treatment of diabetic patients with peripheral neuropathy focuses on ulcer prevention to limit the associated morbidity and mortality risks. Prevention remains the gold standard for reducing DFUs. Common treatment regimens involve blood sugar management, weight loss, tobacco cessation, proper shoe wear and/or frequent skin inspections. However, despite an adherence to a comprehensive ulcer prevention program, a diabetic ulcer may still occur.

Inventors have discovered many technical inefficiencies and shortcomings related to the monitoring and management of the neuromuscular gait, stance, and podiatric performance associated with end users with various podiatric issues. Embodiments of the present disclosure have been designed and implemented to address these technological inefficiencies and shortcomings and have been detailed herein.

### BRIEF SUMMARY

Embodiments of the present disclosure are directed to a portable podiatry sensing platform with a recommendation and feedback system that can be tailored to particular regions of the feet of an end user. Embodiments herein have been termed the Smart Adjustable FEet activity tracking and Recommendation (SAFER) system. The main components of the SAFER system include podiatric data central (PODAC) module, movable podiatry tracker (MOPT), and an external power unit. The central hub is contained in the PODAC module, which handles primary communication with an end device of the end user as well as the MOPTs. The data gathered by the MOPTs is summarized by the PODAC module and transmitted to the end device associated with the end user.

The MOPT handles signal processing of podiatry-related sensors, including but not limited to, force sensing resistors, shear force sensors, and/or temperature and humidity sensors. In some embodiments, sensors such as the force sensing resistors and the shear force sensors require additional circuitry in the form of signal processing circuits. MOPTs can be designed with multiple arms configured to spread out the sensors to create a better spatial measurement map.

Additionally, an active feedback unit inside a respective MOPT can help guide the end user by using vibration, heat, sound, and/or other types of alerts based at least in part on the appropriateness of the alert relative to the foot activity and condition of the end user. The PODAC module and the MOPTs are built in multiple layers, which include electronic-safe epoxy, surface mount components, flexible PCB substrate, and/or waterproof/skin-safe adhesives. The inclusion of certain layers in the PODAC module and/or MOPTs is dependent on the intended surfaces such as, for example, existing footwear and/or skin (epidermis), as well as the location of the PODAC module and/or MOPTs. For example, in some embodiments an MOPT can be stuck on top of the surface or embedded within the footwear. The flexible and portable design of the PODAC and/or MOPTs enable the end user, professionals, and/or manufactures to place these sensors in particular regions of the foot for personalized monitoring.

A mobile software application associated with an end device (e.g., a smartphone) associated with the end user integrates with an information database configured to store present and previous state-of-foot conditions determined based at least in part on data generated by the PODAC modules and/or the relative locations of the MOPTs. Data measured, gathered, calculated, and/or otherwise obtained by the PODAC module and/or MOPTs can be used in a foot heatmap algorithm, a self-calibration procedure, and/or a recommendation system configured to provide active feedback to the end device associated with a particular end user. The recommendation system configured to provide active feedback notifies, guides, and/or advises the end user on their foot activity. The recommendation system can generate one or more recommendations for a particular end user comprising recommendations to make adjustments to footwear and/or stance, to take rests to avoid overexertion, and/or to alter the gait of the end user in order to mitigate potential damage to the foot and encourage best practices. Embodiments of the present disclosure can be applied to multiple use cases including, but not limited to, localized foot ulcer monitoring for certain patients and/or foot performance monitoring for athletic and fitness applications.

As mentioned herein, gait disorders are frequently seen in the context of neuromuscular disorders. The most common neurologic causes include Parkinson's disease, frontal gait disorders, cerebral ataxia, and spasticity due to stroke, multiple sclerosis, or spinal cord injury. For a number of these gait disturbances, the first line of treatment is non-surgical. Treatment modalities include medications to decrease muscle tonicity, physical therapy, and bracing. In this patient population, bracing has been shown to be effective, however, there are risks. Modern braces are designed to fit more intimately than in the past. Although this has led to improved efficacy, there is an increase in the incidence of lower extremity skin issues related to structural or environmental stresses. Embodiments of the present disclosure can alert a physician and/or a patient of the risk of skin compromise in real-time by monitoring moisture, temperature, and/or pressure, as well as other parameters.

Traditional gait analysis for athletes focuses on how the athletes walk and/or run. The information obtained from the gait analysis can provide feedback on the body mechanics and running style of a particular athlete. The gait analysis evaluates the biomechanics of how skeletomuscular joints move in motion in order to diagnose poor running patterns and/or prevent injury. For high performance athletes and weekend warriors alike, gait analyses are often performed in a laboratory by specialists who are focused on the proper alignment and weight distribution of the body of the athlete in order to prevent injury and improve athletic efficiency. However, once the athlete leaves the laboratory, the gait analysis is over. Embodiments of the present disclosure allow continuity of feedback for gait analysis in the real-world environment by directly presenting the feedback to the athlete via an associated end device (e.g., a smartphone associated with the athlete).

As mentioned herein, the CDC estimates that 37.3 million people in the United States are diabetic and nearly 50% of diabetics suffer from some component of peripheral neuropathy (damaged nerves which impair sensation). Diabetic foot ulcers (DFU) are a serious complication of peripheral neuropathy that results in significant morbidity and mortality. Mortality rates associated with the development of a DFU are estimated to be 5% in the first 12 months, and 5-year mortality rates have been estimated at 42%. In addition, patients with DFUs were also found to have a 2.5× increased risk of death compared with their diabetic counterparts without foot wounds. Considering these findings, the medical treatment of diabetic patients with peripheral neuropathy focuses on ulcer prevention to limit the associated morbidity and mortality risks. Prevention is the gold standard for reducing DFUs. Traditional treatment regimens involve blood sugar management, weight loss, tobacco cessation, proper shoe wear and/or frequent skin inspections.

However, despite an adherence to a comprehensive ulcer prevention program, a diabetic ulcer may still occur. In the presence of an ulcer, removing the pressure from the wound or “wound offloading” is the next best step toward management. Plantar shear stress, which is the horizontal component of ground reaction forces, and, to a lesser degree, vertical plantar pressure are major causative factors in the development and poor healing of DFUs. Relieving plantar pressure and shear stress from a DFU is a vital part of wound care, as it promotes healing and prevents recurrence. Wound off-loading can be achieved by many mechanisms, including shoe modifications, specialized boots, and/or orthotic walkers. Nonetheless, compliance with wound off-loading instructions is noted to be poor in the diabetic population. The neuropathic absence of pain likely makes it easy for patients to convince themselves that they are not significantly injuring their feet during intermittent times of noncompliance. Lack of education may also contribute to non-compliant behavior. To correct this and other problems, embodiments of the present disclosure monitor multiple parameters under an at-risk area of the foot while providing instant feedback (e.g., via a smartphone associated with an end user) to help improve compliance with treatment. Embodiments will also, by extension, promote ulcer prevention and/or healing, as well as assist clinicians with treatment plans.

### BRIEF DESCRIPTION OF THE DRAWINGS

Some embodiments of this present disclosure may be further understood by the detailed descriptions and corresponding figures.

FIG. 1 illustrates a smart adjustable feet activity tracking and recommendation (SAFER) system overview according to an example embodiment of the present disclosure;

FIG. 2 illustrates a SAFER system architecture according to an example embodiment of the present disclosure;

FIG. 3 illustrates a moveable podiatry tracker (MOPT) configured for adhering to the surface of an existing footwear and/or skin according to an example embodiment of the present disclosure;

FIG. 4 illustrates an MOPT configured to be embedded beneath the surface of an existing footwear according to an example embodiment of the present disclosure;

FIG. 5 illustrates a podiatric data central (PODAC) module configured according to an example embodiment of the present disclosure;

FIG. 6 illustrates an MOPT system architecture configured according to an example embodiment of the present disclosure;

FIG. 7 illustrates an MOPT configured with an extension arm comprising sensors according to an example embodiment of the present disclosure;

FIG. 8 illustrates an MOPT configured with multiple extension arms comprising respective sensors according to an example embodiment of the present disclosure;

FIG. 9 illustrates a signal conditioning circuit according to an example embodiment of the present disclosure;

FIG. 10 illustrates an active feedback unit according to an example embodiment of the present disclosure;

FIG. 11 illustrates a data flow associated with an end device according to an example embodiment of the present disclosure;

FIG. 12 illustrates a heatmap output for a system with two MOPTs per PODAC module according to an example embodiment of the present disclosure;

FIG. 13 illustrates a threshold-based feedback approach according to an example embodiment of the present disclosure;

FIG. 14 illustrates a pattern-based feedback approach configured to predict current and/or future foot activity patterns according to an example embodiment of the present disclosure;

FIG. 15 illustrates a flowchart diagram associated with the recommendation system with active feedback associated with the SAFER system according to an example embodiment of the present disclosure;

FIG. 16 illustrates an exemplary deployment of the SAFER system on a foot ulcer patient according to an example embodiment of the present disclosure;

FIG. 17 illustrates an exemplary deployment of the SAFER system on a running athlete to monitor performance of particular regions of the feet of the running athlete according to an example embodiment of the present disclosure;

FIG. 18 illustrates a block diagram of an apparatus that can be employed as an end device according to an example embodiment of the present disclosure; and

FIG. 19 illustrates a flowchart diagram for monitoring the condition and performance of the feet of a particular end user according to an example embodiment of the present disclosure.

### DETAILED DESCRIPTION

Some embodiments of the present invention will now be described more fully hereinafter with reference to the accompanying drawings, in which some, but not all, embodiments of the invention are shown. Indeed, various embodiments of the invention may be embodied in many different forms and should not be construed as limited to the embodiments set forth herein; rather, these embodiments are provided so that this disclosure will satisfy applicable legal requirements.

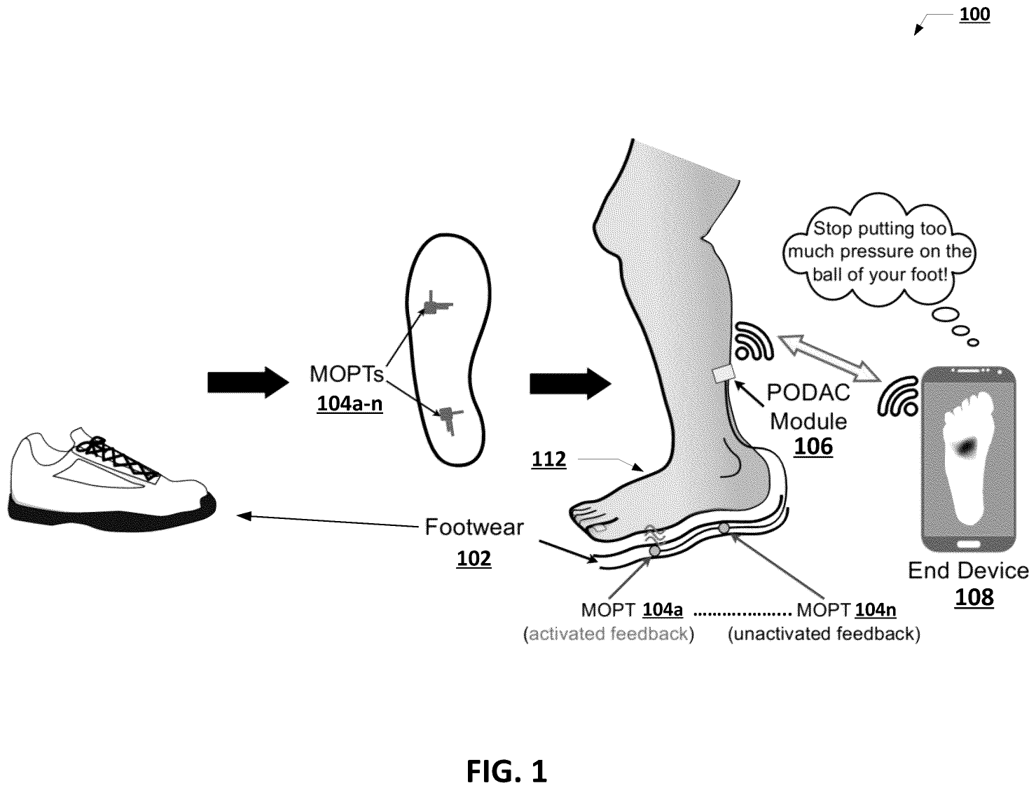

Embodiments herein have been termed the Smart Adjustable FEet activity tracking and Recommendation (SAFER) system. FIG. 1 illustrates an example SAFER system 100 in accordance with one or more embodiments of the present disclosure. The SAFER system 100 is a localized foot monitoring IoT sensor platform configured to track podiatry-related parameters related to one or more feet 112 associated with an end user. As shown in FIG. 1, The SAFER system 100 is configured to gather, aggregate, analyze, and/or otherwise process data associated with the one or more feet 112 such as, for example, normal and shear forces as well as temperature, and provide active feedback via an end device 108. The SAFER system 100 works with existing footwear 102 (e.g., shoes or cast) of the end user.

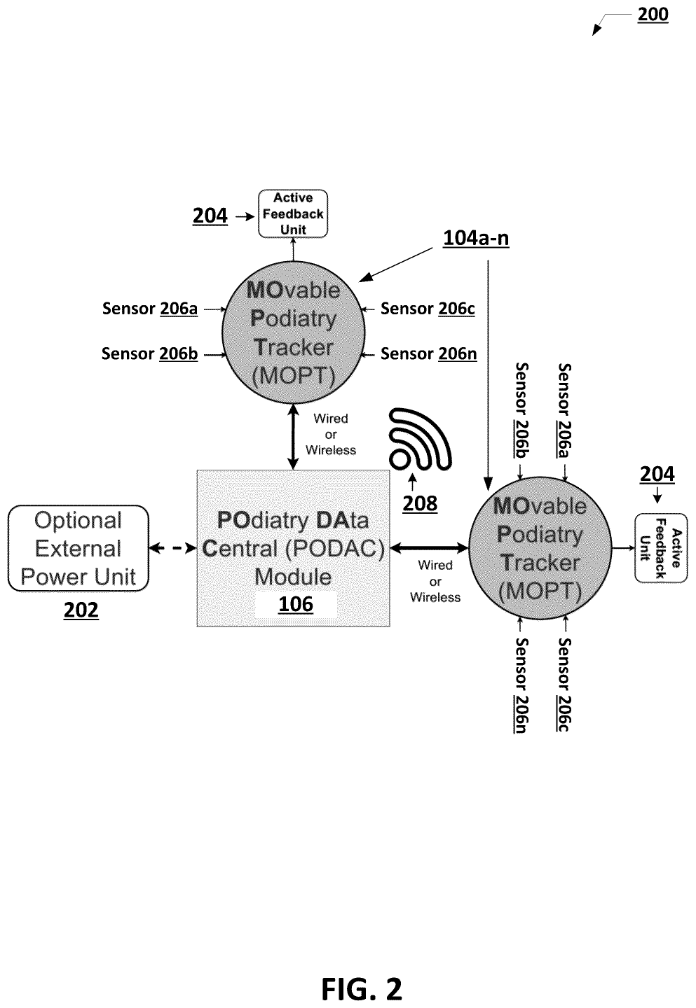

FIG. 2 illustrates the general architecture of the SAFER system 100. The central module of the SAFER system 100 is the POdiatric DAta Central (PODAC) module 106 which features a wireless module 208 for communicating with the end device 108. In various embodiments, the end device 108 can be a smartphone, computer, tablet, cloud-based application, and/or the like that is configured to process and/or render one or more portions of data related to the SAFER system 100. Attached to this PODAC module 106 are MOvable Podiatry Trackers (MOPTs) 104a-n, which contain multiple podiatry-related sensors 206a-n and an active feedback unit 204. The MOPTs 104a-n are configured to minimize foot ulcers by utilizing flexible PCB materials (e.g., such as polyimide) to conform to and/or be embedded within the footwear 102 (e.g., within a shoe insole, wall, etc.) of an end user.

In various embodiments, MOPTs 104a-n can be designed in various respective configurations. For example, in some embodiments, the MOPTs 104a-n can be configured with one or more extended arms for housing each of the respective sensors 206a-n. The designs of the MOPTs 104a-n are highly configurable and can suit different arm lengths and/or geometries in order to create a better spatial measurement map of one or more particular feet 112. The active feedback unit 204 in each MOPT 104a provides haptic, voice, and/or other guiding feedback to the end user. In various embodiments, the active feedback unit 204 engages based at least in part on the activity of the end user and/or one or more parameter values recorded by the whole sensor platform.

In one or more embodiments, the sensors 206a-n are configured to execute a self-calibration process. The self-calibration process of the sensors 206a-n uses data from each of the respective sensors 206a-n as well as known references in order to account for the aging of the sensors 206a-n. The self-calibration process also offers the technical benefit of increasing reliability of the SAFER system 100 throughout the lifetime of the sensors 206a-n. The SAFER system 100 is intended to be configured as a portable unit that is powered using solid-state and/or lithium-ion batteries. Such battery types can be comprised within the optional external power unit 202 and provide the benefit of wireless power so that an end user must not be required to use a wired connection at a specialized environment.

The SAFER system 100 is configured to connect wirelessly using various communication standards (e.g., Bluetooth, Zigbee, Wi-Fi, and/or the like) to an end device 108 (e.g., a smartphone or cloud-based application) that enables the end user, medical personnel, and/or fitness professionals to access podiatry-related data. Using the SAFER system 100, the end user has control over an information database comprising data related to the respective feet activity associated with the end user. Furthermore, an end user can upload the respective feet activity data to the cloud to allow one or more trusted professionals (e.g., medical personnel and/or fitness professionals) to gain remote access to the respect feet activity data. The mobile software applicate integrated with the end device also recommends feet usage and warns the end user to rest their feet if they are causing potential damage to them based at least in part on their activity.

A mobile software application associated with the SAFER system 100 is configured to integrate with the active feedback unit 204 to provide guidance regarding the foot activity (e.g., related to the foot 112) of a particular end user in order to encourage best treatment practices and/or to discourage activity that can cause injuries or further damage the respective foot 112. Based at least in part on the placement of each of the MOPTs 104a-n on the foot 112 and/or footwear 102 of an end user, the mobile software application associated with the SAFER system 100 can generate respective heatmap representing various parameters (e.g., pressure, temperature, humidity, and/or the like). The heatmap functionality of the mobile software application associated with the SAFER system 100 reflects the activity of one or more portions of the foot 112 and can integrate with a recommendation system associated with the SAFER system 100 to facilitate computing footrest time and/or other relevant health concern mitigation techniques associated with the foot 112 of an end user.

### Various Designs of the Embodiments of the Present Disclosure

### Hardware/Material Layers

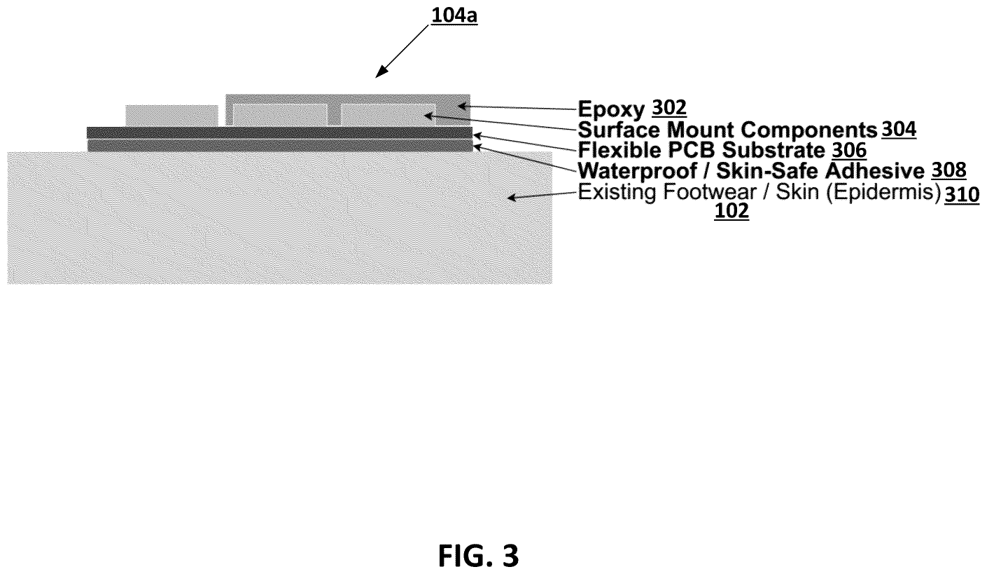

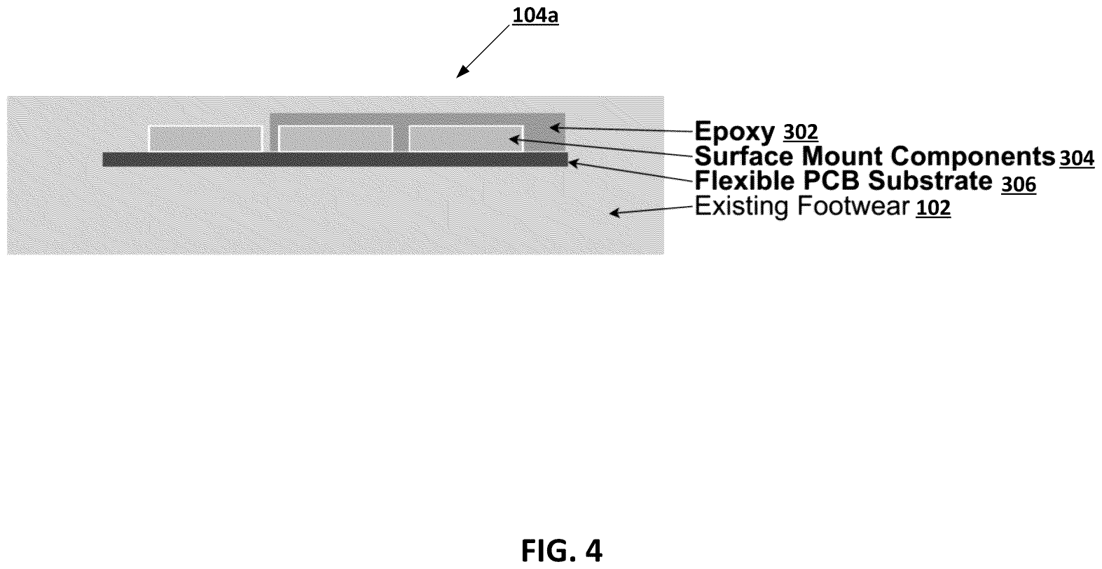

Each component of the SAFER System 100 is constructed using multiple material layers to support the circuitries of the various components (e.g., the PODAC module 106 and/or the MOPTs 104a-n) within an existing footwear 102 or the skin (epidermis) of the end user while retaining their comfort. The material layer design includes, but is not limited to, two primary embodiments of this multi-layered design. The first embodiment of the multi-layered design is illustrated in FIG. 3. As shown in FIG. 3, the first embodiment of the multi-layered design features a configuration in which electronic components are fixed on top of an intended surface such as, for example, the surface of an existing footwear 102 and/or the skin (epidermis) 310 of a particular end user. The second embodiment of the multi-layered design is illustrated in FIG. 4. As shown in FIG. 4, the second embodiment of the multi-layered design features a configuration in which the electronic components are embedded within the footwear 102 (e.g., embedded in a shoe insole).

As depicted, both embodiments of the multi-layered design detailed in FIGS. 3 and 4 feature similar layers that can be used to construct the various components of the SAFER system 100 (e.g., the MOPTs 104a-n and/or or PODAC module 106). For example, the electronic components of both embodiments depicted in FIGS. 3 and 4 utilize a flexible printed circuit board (PCB) substrate 306 with surface mount components 304. The flexible PCB substrate 306 uses flexible materials such as polyimide or clear plastic. In order to mitigate unintended foot ulcers, through-hole components are not used in these embodiments. For example, one or more pins protruding from an integrated chip (IC) package may exacerbate foot ulcers. Surface mount components 304 are generally smaller than through-hole components and allow for a reduced PCB area footprint. Deploying the reduced PCB area footprint also provides the added benefit or reducing irritation and/or further exacerbating an existing foot ulcer.

As shown in FIGS. 3 and 4, the electrical layers of the various embodiments of the components of the SAFER system 100 (e.g., the MOPTs 104a-n and/or the PODAC module 106) can be structurally fortified using electronic-safe epoxy 302 that goes on top of the flexible PCB substrate 306 and the surface mount components 304. The addition of the electronic-safe epoxy 302 makes the various components waterproof and helps the various components to better conform to the intended surface (e.g., the skin 310 and/or the existing footwear 102). The electronic-safe epoxy 302 also helps the various components of the SAFER system 100 resist mechanical stress. For example, the electronic-safe epoxy 302 can fortify a respective MOPT 104a that an end user repeatedly steps on.

One factor that can be considered when selecting the first or second embodiments of the multi-layered designs (e.g., as provided in FIGS. 3 and 4 respectively) for a particular application and/or end user is the amount of room inside the existing footwear 102. Some surface mount components 304 prohibit the application of the electronic-safe epoxy 302 such as, for example, certain sensors 206a-n (e.g., force sensors) which may already have inherent structural integrity and therefore do not require the additional electronic-safe epoxy 302. Such sensors 206a-n can be placed closer to the edge of the flexible PCB substrate 306 edge without the need for the electronic-safe epoxy 302 on top. In certain embodiments, before skin application on certain designs, a waterproof/skin-safe adhesive 308 is applied to the bottom of the flexible PCB substrate 306 (e.g., as depicted in FIG. 3). In various embodiments, the waterproof/skin-safe adhesive 308 can be replaced after repeated uses. In scenarios in which a component (e.g., an MOPT 104a) is embedded into the footwear 102 (e.g., as shown in FIG. 4), the component (e.g., the MOPT 104a) can be adhered to the footwear 102 using waterproof adhesives and/or can be inserted directly into the inside of the footwear 102.

### Podiatric Data Central (PODAC) Module

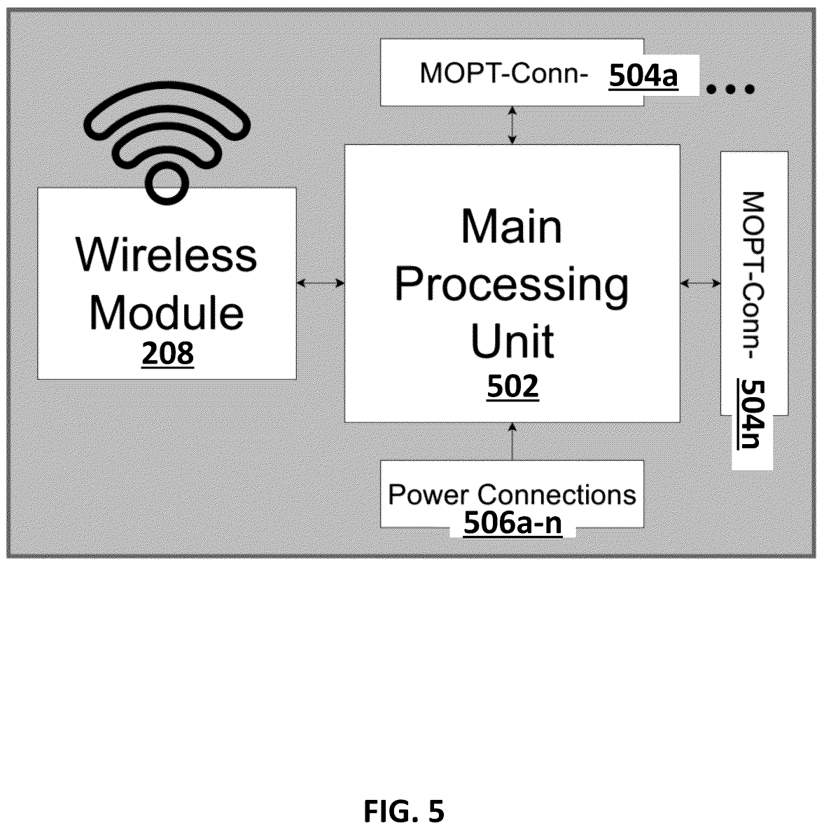

FIG. 5 illustrates a podiatric data central (PODAC) module 106 configured according to an example embodiment of the present disclosure. The PODAC module 106 can be understood as the “brain” of the SAFER system 100. The PODAC module 106 acts as the main processing unit and communicates with the MOPTs 104a-n and the wireless module 208. The PODAC module 106 dedicates a separate communication channel for the onboard wireless module 208, so the wireless module 208 does not get interfered with by other components of the PODAC module 106 connected by wired communications interfaces such as, for example, the power connections 506a-n. The main processing unit 502 aggregates and/or configures sensor data (e.g., collected by sensors 206a-n) from one or more MOPTs 104a-n into summary data before sending the summary data wirelessly (e.g., via the wireless module 208) to the end device 108 (e.g., a smart-phone, computer, and/or cloud-based application associated with an end user). In various embodiments, the MOPTs 104a-n can be configured to communicate with the PODAC module 106 via wired and/or wireless communications. The PODAC module 106 uses a shared bus with all the MOPTs 104a-n using serial communications interface (e.g., a serial peripheral interface, integrated circuit, and/or the like) when communicating via a wired connection. The MOPT connectors 504a-n can be deployed in various configurations (e.g., magnetic connectors, ribbon connectors, edge connectors, and/or the like) and serve as the connection points to link multiple MOPTs 104a-n to a single PODAC module 106. In various embodiments, the MOPT connectors 504a-n can be omitted when using wireless connectivity in the electrical design for both the MOPTs 104a-n and PODAC module 106. Such wireless embodiments provide the technical benefit of further shrinking the PCB area footprint.

### Movable Podiatry Tracker (MOPT)

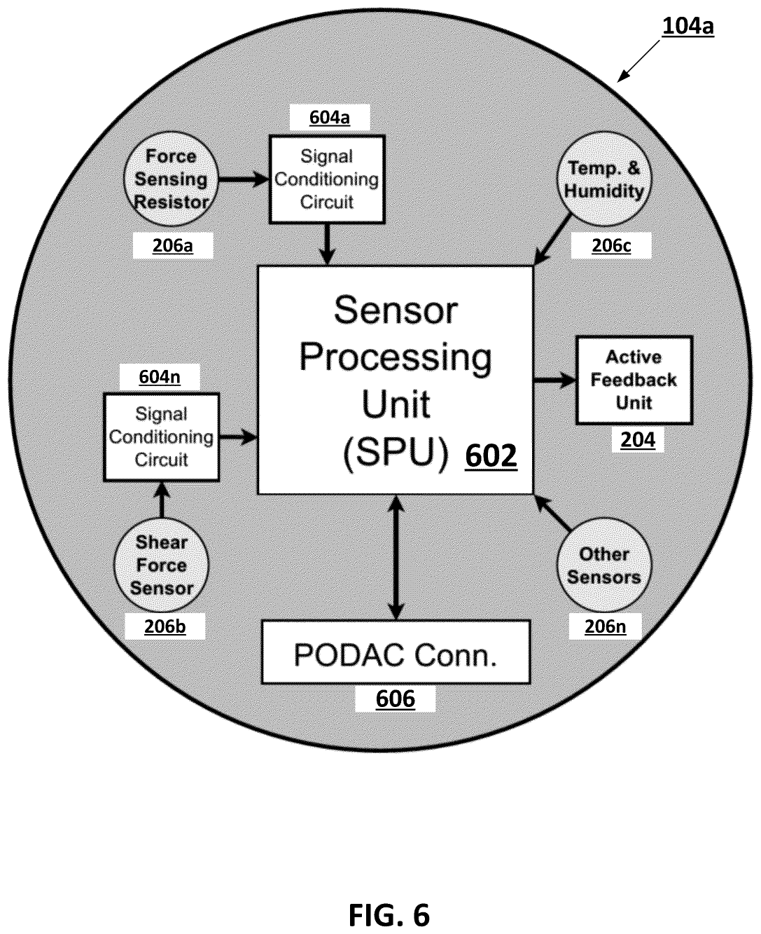

FIG. 6 illustrates a system architecture for an example movable podiatry tracker (MOPT) 104a configured according to an example embodiment of the present disclosure. The MOPT 104a is a portable monitoring peripheral unit that collects raw podiatry-related data from connected sensors 206a-n and transmits the raw podiatry-related data to the PODAC module 106 for data processing before the data gets transmitted to the end device 108. MOPTs 104a-n are constructed in a similar fashion as the PODAC module 106 in order to maintain conformity with the intended surface (e.g., embedded with or on top of the existing footwear 102). As shown in FIG. 6, an MOPT 104a comprises a sensor processing unit 602, a wired PODAC connector 606 (if applicable), one or more podiatry-related sensors 206a-n, and/or one or more signal conditioning circuits 604a-n (if applicable).

The MOPTs 104a-n are designed to be placed anywhere on, or within, an existing footwear 102 (e.g., embedded in the insole of the footwear 102) to monitor a particular part of the foot 112. The podiatry-related data gathered by one or more MOPTs 104a-n guides the recommendation, active feedback, and/or self-calibration software systems at the end device 108. The podiatry-related data gathered by the one or more MOPTs 104a-n is also configured to facilitate the generation of heatmap associated with the foot 112 of a particular end user so that the particular end user better understands how corresponding foot activity affects particular regions of the foot 112. In various embodiments, an automated algorithm can determine the MOPTs 104a-n placement using visual data (e.g., image data captured by a camera associated with the end device 108) and/or other means. The visual data (e.g., image data) can be configured to facilitate the visualization of the respective placement of MOPTs 104a-n relative to the foot 112 of a particular end user.

The sensor processing unit 602 is a low-power microprocessor with multiple analog-to-digital converters (ADCs) to translate raw analog sensor data into digital data with relevant units. The sensor processing unit 602 can employ dedicated channels for various sensors 206a-n that utilize a serial communications interface. As described previously, the MOPT 104a uses a physical connection interface (e.g., magnetic connectors, ribbon connectors, and/or the like). Examples of podiatry-related sensors 206a-n can include but are not limited to force sensing resistor (FSR), shear force sensor (SFS), temperature, and/or humidity sensors. It will be appreciated that other podiatry-related sensors can be easily integrated into the design of one or more MOPTs 104a-n while maintaining the features described herein by utilizing the multi-layered material design. The FSR, SFS, and other sensors 206a-n that produce small output signals may require the use of one or more signal conditioning circuits 604a-n. The one or more signal conditioning circuits 604a-n are configured to preprocess the raw analog signals from the sensors 206a-n in order to output manageable analog signals that the analog-to-digital converters (ADCs) from the sensor processing unit 602 can properly digitize. The one or more signal conditioning circuits 604a-n can be calibrated depending on the minutia of the manufacturing processes of various FSRs and SFSs. In various embodiments, other sensors 206a-n feed directly into wired communications interfaces or ADCs of the sensor processing unit 602. In addition to the sensors 206a-n, the sensor processing unit 602 interfaces with the active feedback unit 204 to inform the end user about the effect of the corresponding foot activity of the foot 112. Information related to the foot activity of the foot 112 can comprise information related to both good and bad practices related to the used of the foot 112. For example, if a particular end user has an ulcer associated with foot 112 and is not resting the foot 112 enough, information related to the bad activity related to the foot 112 can be generated and transmitted to the end device 108.

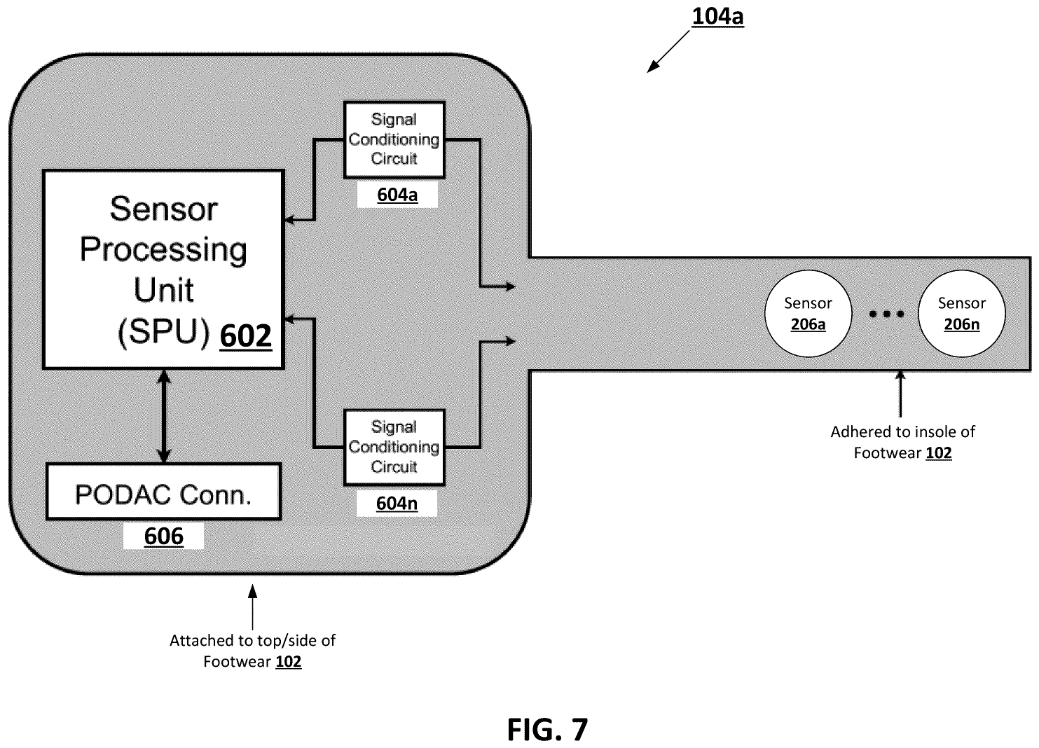

One or more MOPTs 104a-n utilize the system architecture provided in FIG. 6 while be deployed in various configurations. For example, FIG. 7 illustrates an MOPT 104a configured with an extension arm comprising sensors 206a-n. In such an embodiment, the connected sensors 206a-n are placed on a single arm extending from the main part of the MOPT 104a which comprises the sensor processing unit 602 and the signal conditioning circuits 604a-n. The main part of the MOPT 104a can be placed on the top or sides of the inside of the existing footwear 102 to ensure that an end user does not step on the main part of the MOPT 104a. The single arm extending from the main part of the MOPT 104a can be situated into the existing footwear 102 such that only a small PCB area footprint is in contact with the sole of the foot 112 of the end user. Such an embodiment ensures minimal contact with the foot 112 to reduce irritation and/or exacerbation of ongoing health issues related to the foot 112.

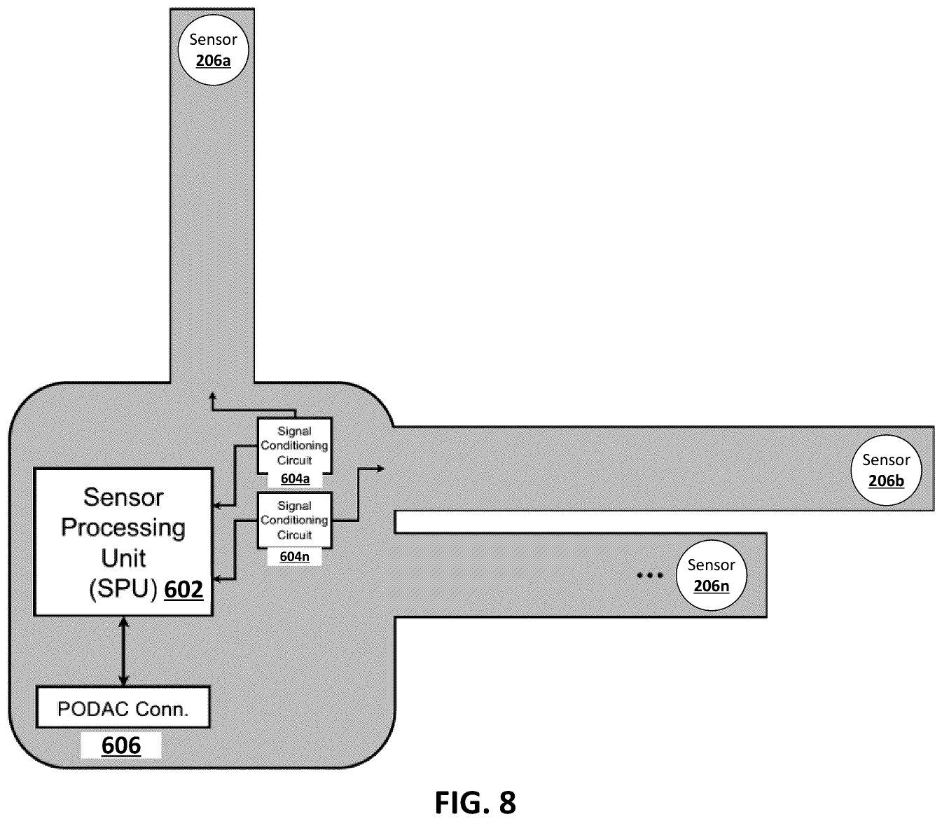

As shown in FIG. 8, another MOPT 104a embodiment can include multiple arms extending from the main part of MOPT 104a. In such an embodiment, each arm can comprise at least one connected sensor 206a-n. Multiple such arms connected to the main part of the MOPT 104a can facilitate the generation of an improved spatial force map associated with a target region of the foot 112. Variations of this multi-arm embodiment can be implemented with various configurations. For example, various multi-arm embodiments can feature different respective lengths and/or geometries. It will be appreciated that the flexibility of the design of such embodiments of the MOPT 104a can be tailored to the specific health and/or fitness monitoring needs of an end user.

### Signal Conditioning Circuit

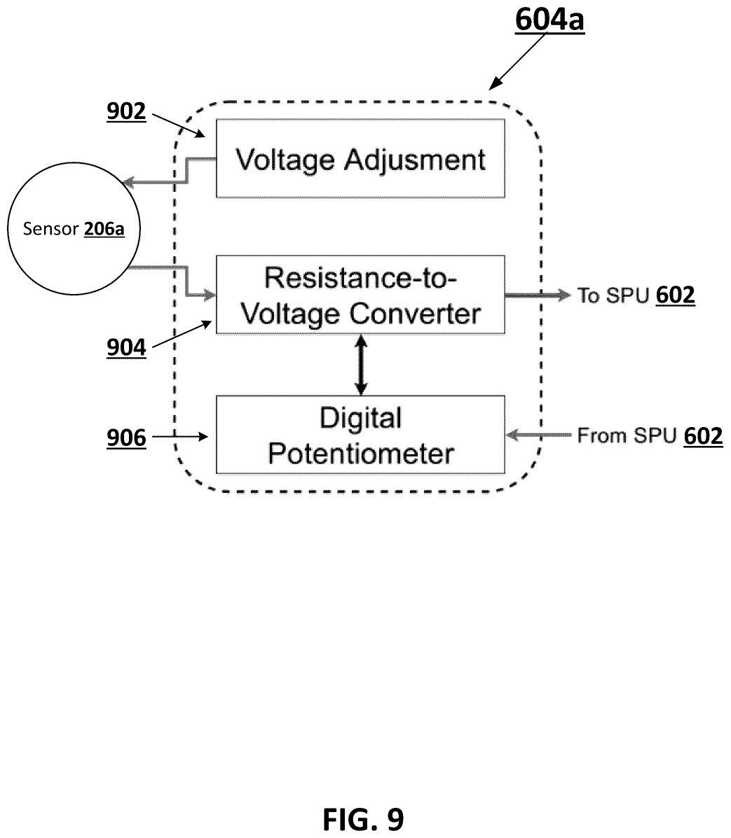

In various embodiments, one or more signal conditioning circuits 604a-n can be connected to the sensors 206a-n of a respective MOPT 104a. FIG. 9 illustrates the component parts of a signal conditioning circuit 604a. The signal conditioning circuit 604a comprises a voltage adjustment circuitry 902, a resistance-to-voltage converter 904, and/or a digital potentiometer 906. The signal conditioning circuit 604a facilitates signal pre-processing for potentially weak signals generated by one or more sensors 206a-n associated with an MOPT 104a. For example, various sensors 206a-n such as force sensing resistors and/or shear force sensor may generate weak signal output. Signals that are pre-processed by the signal conditioning circuit 604a can be transmitted to the analog-to-digital converter (ADC) of a sensor processing unit 602 for further processing and data collection.

The voltage adjustment circuitry 902 is configured to take the power supply voltage and transform the voltage appropriately for the sensors 206a-n to enable the sensors 206a-n to work in tandem with the resistance-to-voltage converter 904. The resistance-to-voltage converter 904 is configured to transform a resistance value that is observed from the sensors 206a-n to a voltage that is within the range that the ADC of a respective sensor processing unit 602 can handle. The resistance-to-voltage converter 904 can be implemented by various devices including, but not limited to, operational amplifiers associated with the resistance-to-voltage converter 904. Determined by the output signal from the resistance-to-voltage converter 904, the sensing accuracy of a sensor 206a can be finely calibrated using a digital potentiometer 906 controlled by the sensor processing unit 602. In various embodiments, the digital potentiometer 906 controls the feedback resistance of an operational amplifier within the resistance-to-voltage converter 904. The value of the digital potentiometer 906 can be adjusted during the initial calibration phase of a corresponding MOPT 104a as well as during subsequent adjustment calibrations executed throughout the lifetime of the corresponding MOPT 104a.

### Active Feedback Unit

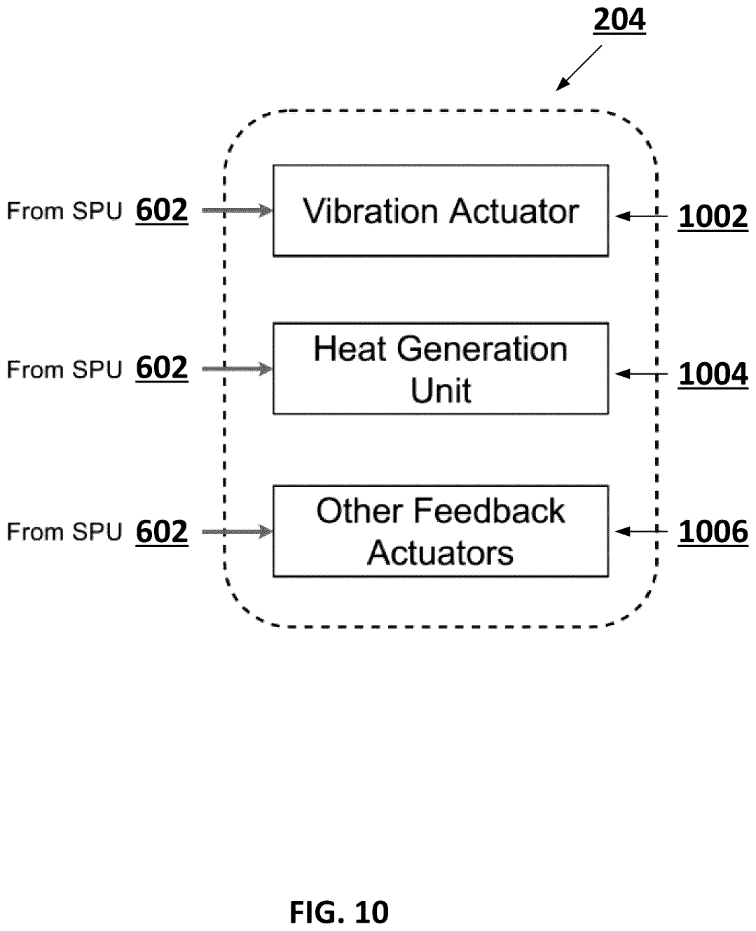

FIG. 10 illustrates an active feedback unit 204 configured according to an example embodiment of the present disclosure The active feedback unit 204 connects to the sensor processing unit 602 of an MOPT 104a to provide guidance to an end user about foot activity associated with a foot 112 of the end user. In various embodiments, the active feedback unit 204 comprises various feedback mechanisms including, but not limited to, a vibration actuator 1002, a heat generation unit 1004, and/or one or more other feedback actuators 1006. The active feedback unit 204 is configured to work in tandem with an end device 108. As such, the end device 108 can provide additional sound and/or visual feedback to an end user associated with the end device 108. Whenever the end user performs an activity (based at least in part on a predetermine threshold or pattern) that can be beneficial or detrimental to the end user, the end device 108 can send a positive or negative indication to the PODAC module 106 and then to the corresponding MOPT 104a that has been designate to the issue the corresponding feedback. The positive or negative indication determines the actuation by the respective feedback mechanism in the active feedback unit 204. For example, for detrimental actions, the vibration actuator 1002 can produce a large vibration, whereas beneficial actions can be signified by one or more small vibrational bursts. Different feedback actuation can be executed to differentiate between the positive (e.g., beneficial) or negative (e.g., detrimental) actions executed by an end user associated with a foot 112 being monitored by the SAFER system 100.

### Mobile Software Application Integration

To keep track of the foot activity related to a particular foot 112 and the locations of the MOPTs 104a-n relative to the foot 112, an information database 1102 at the end device 108 is created that facilitates various operations described herein. For example, the information database 1102 facilitates, in conjunction with the end device 108, the generation of a foot heatmap 1104. The information database 1102 is also configured to integrate with a recommendation system with active feedback 1112 to provide guided active feedback to an end user associated with the end device 108. Furthermore, the data comprised in the information database 1102 is configured to facilitate an active self-calibration procedure 1106 to calibrate one or more sensors 206a-n to read sensor data correctly throughout the operational lifetime of an MOPT 104a.

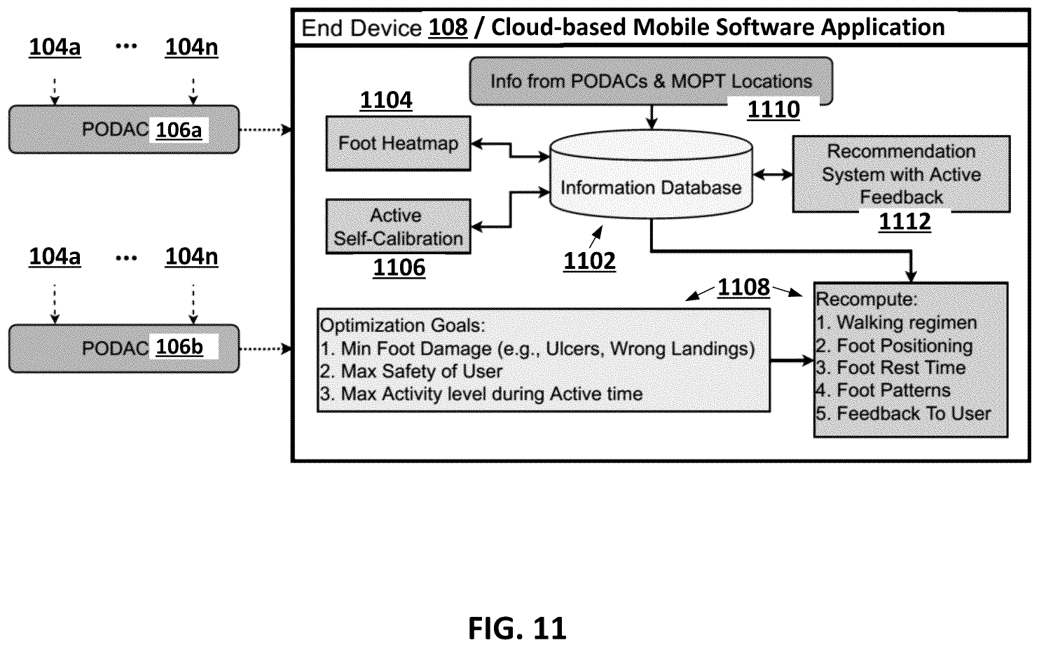

FIG. 11 shows the information flow from the MOPTs 104a-n associated with PODAC modules 106a and 106b to the end device 108 as well as various optimization goals 1108 to be employed during operation of the MOPTs 104a-n and/or the PODAC modules 106a and 106b. Examples of an end device 108 include, but are not limited to, a mobile device (e.g., a smartphone, laptop, tablet, and/or the like) and/or a cloud-based mobile software application. For example, in embodiments in which the information database 1102 and/or other relevant software are located remote from the components of the SAFER system 100, the end device 108 can be a cloud-based mobile software application.

The information database 1102 is configured to store the present state of the sensors 206a-n as well as summary data related to previous states of the sensors 206a-n generated from historical PODAC module information 1110. In various embodiments, the relative locations of the one or more MOPTs 104a-n associated with each respective PODAC module 106 can be manually inputted via an instance of the mobile software application corresponding to the SAFER system 100 associated with a particular end device 108. The relative locations of the one or more MOPTs 104a-n can also be automatically detected by using image data (e.g., image data captured by a camera associated with the end device 108) associated with the existing footwear 102 related to a particular end user. The mobile software application at the end device 108 is configured to employ various image processing and/or machine learning techniques on the image data to determine the position of one or more MOPTs 104a-n associated with the footwear 102 and/or foot 112 of a particular end user. Additionally, or alternatively, various embodiments can determine the location of one or more MOPTs 104a-n based at least in part on sensor data captured by one or more sensors 206a-n during the calibration phase of the SAFER system 100. In various embodiments, the sensor data captured by one or more sensors 206a-n during the calibration phase of the SAFER system 100 can be used in conjunction with various machine learning techniques in order to determine a close approximation of the locations of the MOPTs 104a-n.

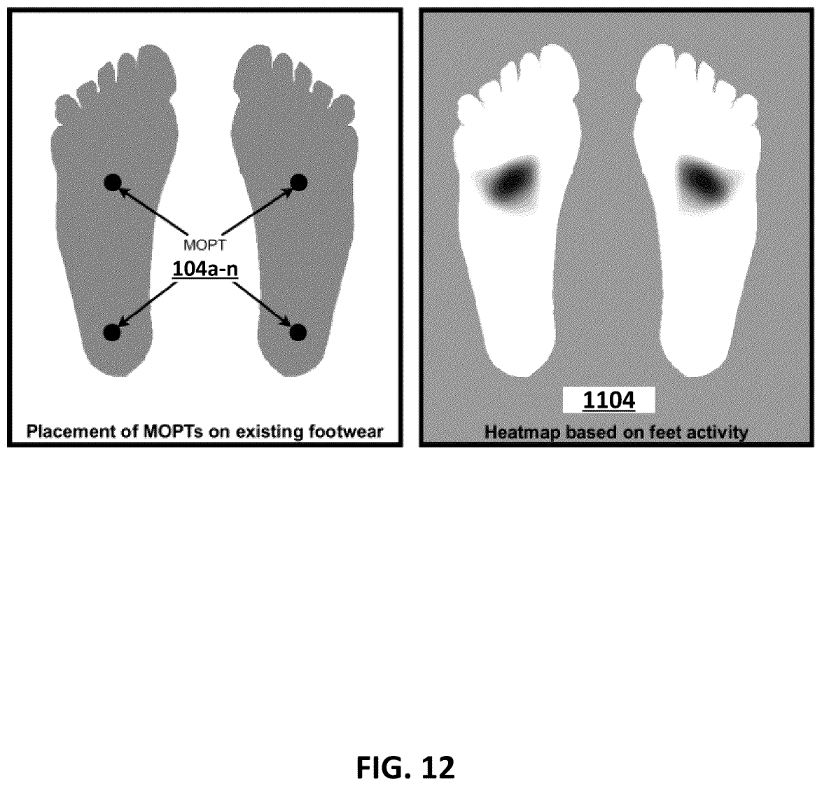

Once the relative locations of the one or more MOPTs 104a-n have been determined, the locations can be inputted into the foot heatmap generation system. FIG. 12 shows an example of heatmap output for a system with two MOPTs 104a-n per PODAC module 106. Lighter regions of the heatmap 1104 indicate that an end user is applying little-to-no pressure on regions of a respective foot 112 associated with the MOPTs 104a-n. Darker shading on the heatmap 1104 indicates greater pressure for those regions of the respective foot 112 associated with the respective MOPTs 104a-n. As such, the heatmap 1104 can be used to determine one or more problem areas associated with a respective foot 112.

As shown in FIG. 12, each PODAC module 106 on each respective foot 112 has one MOPT (e.g., MOPT 104a) near the ball of the foot 112 and one MOPT (e.g., MOPT 104b) near the heel of the foot 112. In this example, based at least in part on the location of the MOPTs 104a-n, it can be seen that the particular end user puts substantial weight onto the bottom of the arch of each respective foot 112 close to the toes of each respective foot 112. As such, it can be understood that the heel of each respective foot 112 poses little-to-no danger to the end user associated with each respective foot 112. The heatmap 1104 can be generated by the end device 108 via the mobile software application associated with the SAFER system 100. The heatmap 1104 can also be used by the recommendation system with active feedback 1112 associated with the SAFER System 100 to facilitate the generation of one or more recommendations configured as notifications to be rendered on the end device 108, where the one or more recommendations can be related to the foot activity of a particular end user.

### Active Self-Calibration

Using the data collected from one or more sensors 206a-n that stored in the information database 1102, an end device 108 can monitor the health of one or more modules within the SAFER system 100 and cause the execution of one or more self-calibration procedures throughout the operational lifetime of the one or more components associated with the SAFER system 100. Active self-calibration procedure 1106 increases the reliability and longevity of the SAFER system 100 throughout its operational lifetime. One example approach for self-calibration is executing referential-based calibration. The end device 108 can ping for external information, such as outside temperature, acceleration, rotation, and/or the like. The external information can inform the calibration of one or more sensors 206a-n associated with one or more respective MOPTs 104a-n. Another example self-calibration approach is cross-calibration in which each respective sensor of the one or more sensors 206a-n helps to calibrate the other sensors 206a-n associated with the one or more respective MOPTs 104a-n. The end device 108 can determine whether a respective sensor of the one or more of the sensors 206a-n is producing a different reading than the other sensors 206a-n based at least in part on the relative locations of the sensors 206a-n, the age of the one or more sensors 206a-n, and/or other relevant conditions associated with the sensors 206a-n. Such information can be stored in the information database 1102.

### Active Feedback

Based at least in part on data comprising in the information database 1102, the recommendation system with active feedback 1112 associated with the end device 108 can provide guidance to an end user based at least in part on whether the end user is performing a foot activity associated with a respective foot 112 in a proper or detrimental manner. The feedback generated by the recommendation system with active feedback 1112 creates a closed-loop recommendation system in which the end user is guided to self-correct various foot actions associated with a respective foot 112 if the end user has been determined to be prone to sustaining injuries and/or causing further damage to a respective foot 112. The recommendation system with active feedback 1112 associated with the end device 108 works in tandem with the active feedback unit 204s inside the one or more MOPTs 104a-n which can provide feedback actuation (e.g., vibration, heat, sound, and/or the like) for guiding the end user on their foot activity. Various embodiments include, but are not limited to, two primary approaches of for generating active feedback via the mobile software application associated with the SAFER system 100 running on a respective end device 108. The first active feedback approach employed in various embodiments is threshold-based feedback in which the readings of each individual sensor 206a-n are compared against referential and/or differential values. The second active feedback approach is pattern-based feedback in which multi-modal sensing data is used to determine a foot activity pattern of a particular end user in order to predict future incidents and/or changes to the foot activity of the particular end user.

### Threshold-Based Feedback Approach

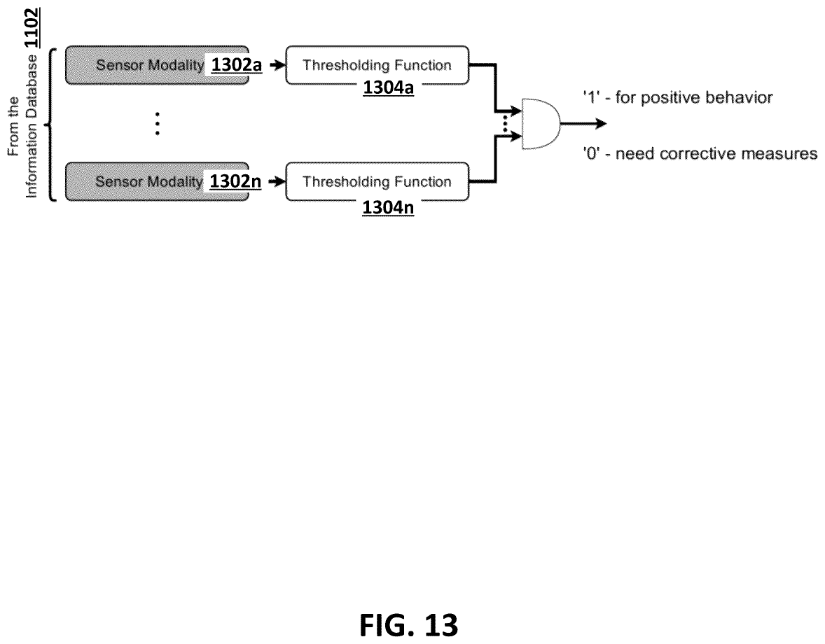

FIG. 13 illustrates a threshold-based feedback approach for generating active feedback according to an example embodiment of the present disclosure Threshold-based feedback considers each individual sensor 206a-n and/or sensor modalities 1302a-n by themselves. The output of each sensor 206a-n that has been stored the information database 1102 is inputted into a respective thresholding function 1304a-n associated with the sensors 206a-n. In such examples, the sensors 206a-n, sensor modalities 1302a-n, and/or the thresholding functions 1304a-n can be calibrated per a particular end user. Thresholding functions 1304a-n can either use reference values or look at the differences in various signals spatially and/or temporally. The output of each thresholding function 1304a-n determines if the respective sensor 206a-n agrees with the general behavior of the end user regarding the respective foot activity associated with a respective foot 112 of the end user. If all sensors 206a-n and/or sensor modalities 1302a-n agree with the general behavior (e.g., that respective foot activity associated with a foot 112 is either positive or detrimental), active feedback related to the general behavior can be transmitted to the end user via the end device 108 and/or the active feedback unit 204 within a respective MOPT 104a.

### Pattern-Based Feedback Approach

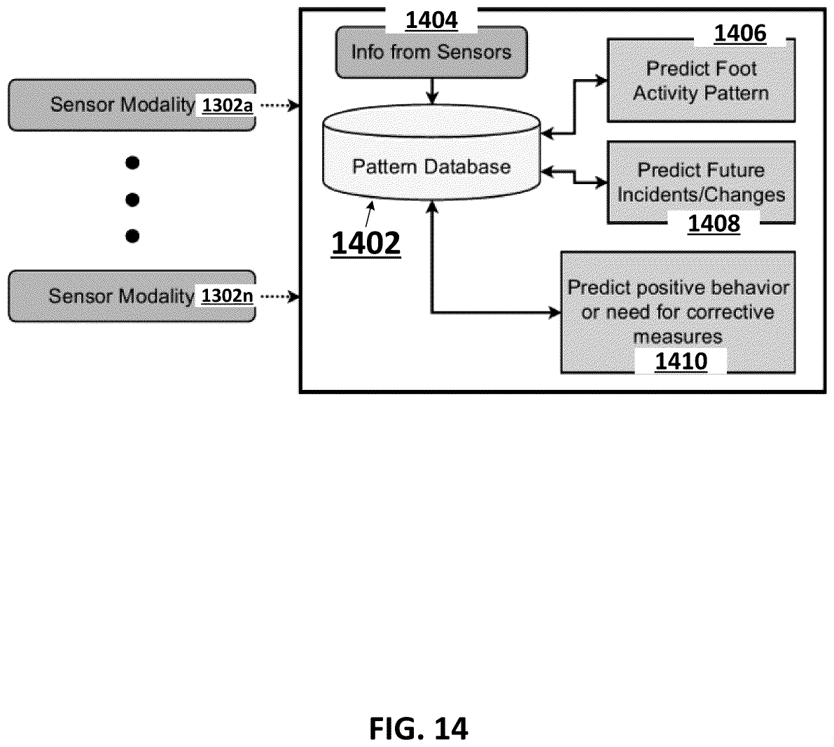

FIG. 14 illustrates a pattern-based feedback approach configured to predict current and/or future foot activity patterns related to a respective foot 112 of a particular end user according to an example embodiment of the present disclosure. The pattern-based feedback approach takes a multi-modal approach to formulating active feedback for the end user about the foot activity of a respective foot 112 associated with the end user. Each portion of information 1404 from the sensor 206a-n and/or sensor modalities 1302a-n coming from the information database 1102 can be compiled together in a pattern database 1402. Based at least in part on the information 1404, the pattern database 1402 can be used to predict the foot activity pattern 1406 of a respective foot 112 associated with an end user. Inference of the current foot activity pattern can also help predict future incidents and/or changes 1408 to foot activity of a respective foot 112. The anticipation of these changes can provide active feedback to help the end user self-correct detrimental foot actions to prevent future injuries and/or further damage to the foot 112 as well as continue to encourage positive behaviors in the future. The pattern database 1402 can be used to predict positive behavior if the end user is performing good practices relative to the respective foot activity or if the end user needs corrective measures 1410 to prevent immediate, future, and/or long-term foot-related injuries. The one or more prediction techniques described herein can be performed using rule-based approaches and/or with the help of artificial intelligence and machine learning models integrated with the end device 108.

Safer System's Recommendation System with Active Feedback

The recommendation system with active feedback 1112 is part of the mobile software application associated with the SAFER system 100 that is executed on one or more end devices 108. Based at least in part on the data generated by one or more PODAC modules 106 stored in the information database 1102 and the foot heatmap output (e.g., heatmap 1104), the recommendation system with active feedback 1112 logs and/or stores certain stages of a particular condition of a respective foot 112 associated with an end user. These stages are configured to facilitate the determination of one or more potentially adverse podiatry issues and/or the generation of one or more potential corrective actions. The recommendation system with active feedback 1112 can also determine positive behaviors regarding the foot activity related to a respective foot 112 associated with a particular end user.

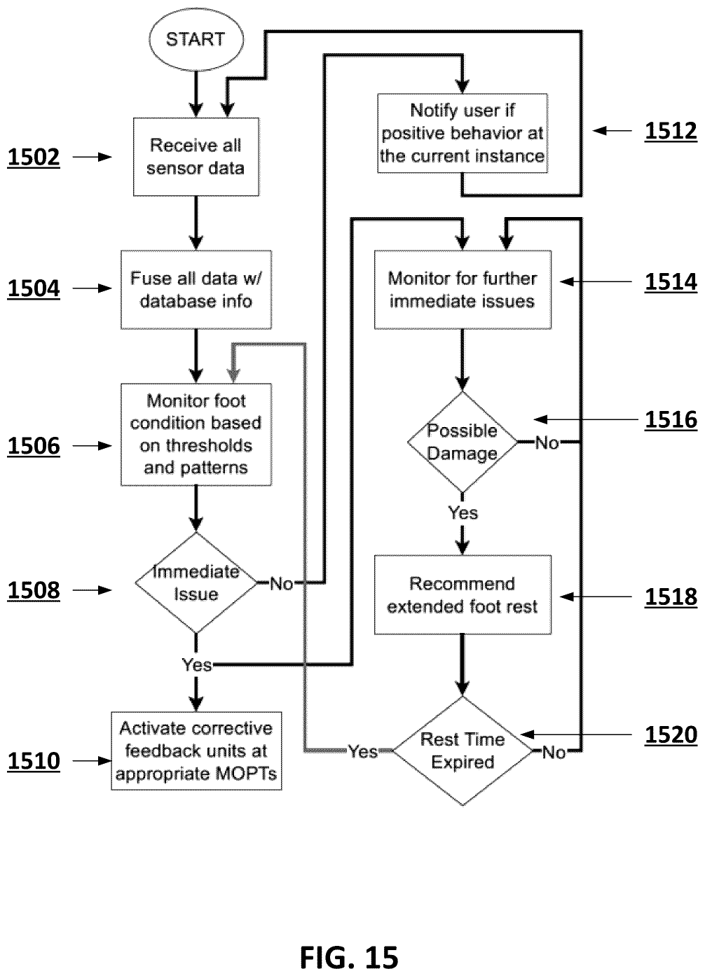

FIG. 15 illustrates a control flowchart associated with the recommendation system with active feedback 1112 as the recommendation system with active feedback 1112 operates via an end device 108. At step 1502, the end device 108 receives all the sensor data aggregated by one or more PODAC module(s) 106, which is collected from sensors 206a-n in the MOPTs 104a-n. After receiving data from the one or more PODAC module(s) 106, at step 1504 the recommendation system with active feedback 1112 fuses all the sensor data along with the data from the information database 1102. At step 1506, the recommendation system with active feedback 1112 using fused data, as well as output data from the heatmap 1104, to monitor the condition of each respective foot 112 and the corresponding regions of interest related to each respective foot 112. At step 1508, the recommendation system with active feedback 1112 checks for any immediate issues related to each respective foot 112. If no immediate issues arise, the recommendation system with active feedback 1112 remains in a no-danger state, and the end device 108 continues to receive sensor data from the one or more PODAC module(s) 106. Additionally, the recommendation system with active feedback 1112 proceeds to step 1512 and the active feedback unit 204 is engaged in order to notify the end user to continue the positive trend of acceptable foot activity in the current instance.

Alternatively, if immediate issues are detected such as, for example, if tremendous pressure on any region of interest associated with a foot 112 is detected, the recommendation system with active feedback 1112 triggers the first level of recommendations in which the recommendation system with active feedback 1112 notifies the end user to case usage of the respective foot 112 and/or to adjust the stance of the respective foot 112. At step 1510, the active feedback unit 204s at the appropriate MOPT 104a locations are activated to guide the end user to self-correct the current detrimental foot activity associated with the respective foot 112. The recommendation system with active feedback 1112 will enter and remain at step 1514 for a certain periodicity of time while the recommendation system with active feedback 1112 continues to monitor for further occurrences of immediate issues and/or potential damage to the foot. At step 1516, possible damage to the respective foot 112 has been detected, the recommendation system with active feedback 1112 will enter step 1518 and recommend extended footrest to the end user for a certain time period. While the end user rests the respective foot 112, the recommendation system with active feedback 1112 will continue to monitor for immediate issues and potential damage whenever the respective foot 112 is being engaged during the rest period. At step 1520, once the prescribed rest period expires, recommendation system with active feedback 1112 will lower the threat level appropriately and continue data collection and/or monitoring of the respective foot 112.

### Example Use Cases of the Embodiments of the Present Disclosure

### Foot Ulcer Localized Monitoring for Certain Patients

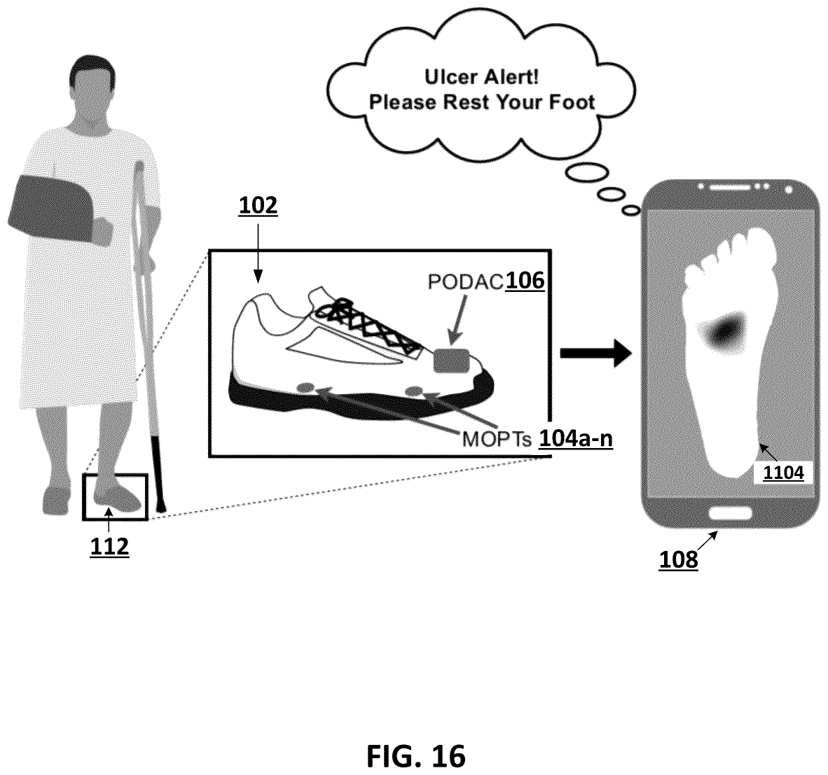

One potential use case for the SAFER system 100 is to monitor for foot ulcers in localized regions of a respective foot 112 of an end user with health conditions including, but not limited to, diabetes, obesity, foot-related injuries, and/or pregnancies. Foot ulcers are caused by placing unnecessary pressure on particular regions of a respective foot 112 which causes blisters and other injuries. In extreme cases, foot ulcers can cause a respective limb of an end user to be amputated. Podiatric physicians are particularly interested in foot ulcers and other foot-related injuries in order to prevent the worst-case scenarios for their patients. FIG. 16 illustrates the deployment of the SAFER system 100 on a patient, focusing on the foot 112. The foot 112 is fitted with the PODAC module 106 on top of the footwear 102 of the patient and the MOPTs 104a-n are embedded within the existing footwear 102. The patient monitors the condition of the foot 112 using the mobile software application associated with the SAFER system 100 that is configured to communicate with the PODAC module 106 and display a heatmap 1104 output on an end device 108 over time. Based at least in part on the foot activity and/or condition related to the foot 112, the recommendation system with active feedback 1112 will alert the patient when it is time to rest the foot 112 in order to mitigate occurrences of foot ulcers.

### Athletic and Fitness Monitoring of Foot Performance

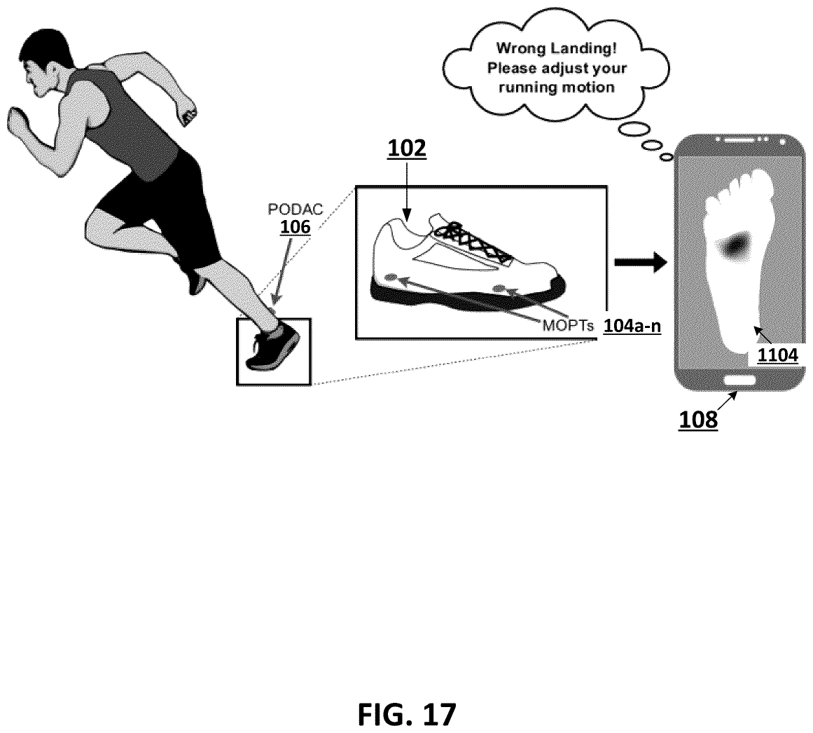

Monitoring the foot performance is another application of the SAFER system 100, and the SAFER system 100 can be configured especially to facilitate the monitoring of each respective foot 112 associated with an athlete. FIG. 17 shows the deployment of the SAFER system 100 on a running athlete to monitor foot performance in certain regions of a foot 112 with MOPTs 104a-n embedded within the existing footwear 102 and a PODAC module 106 adhered to an ankle associated with the running athlete. In this case, the recommendation system with active feedback 1112 monitors not only foot performance but also potential for foot-related injuries due to poor landing and/or running posture. When trouble spots are engaged by the end user, the recommendation system with active feedback 1112 detects a poor landing and provides active feedback to the end user. When poor landing is detected, the recommendation system with active feedback 1112 can notify the running athlete (in this example embodiment) of this issue via the end device 108 and advise the running athlete to adjust the current running motion and/or posture.

### Example Apparatus for Performing Operations of the Present Disclosure

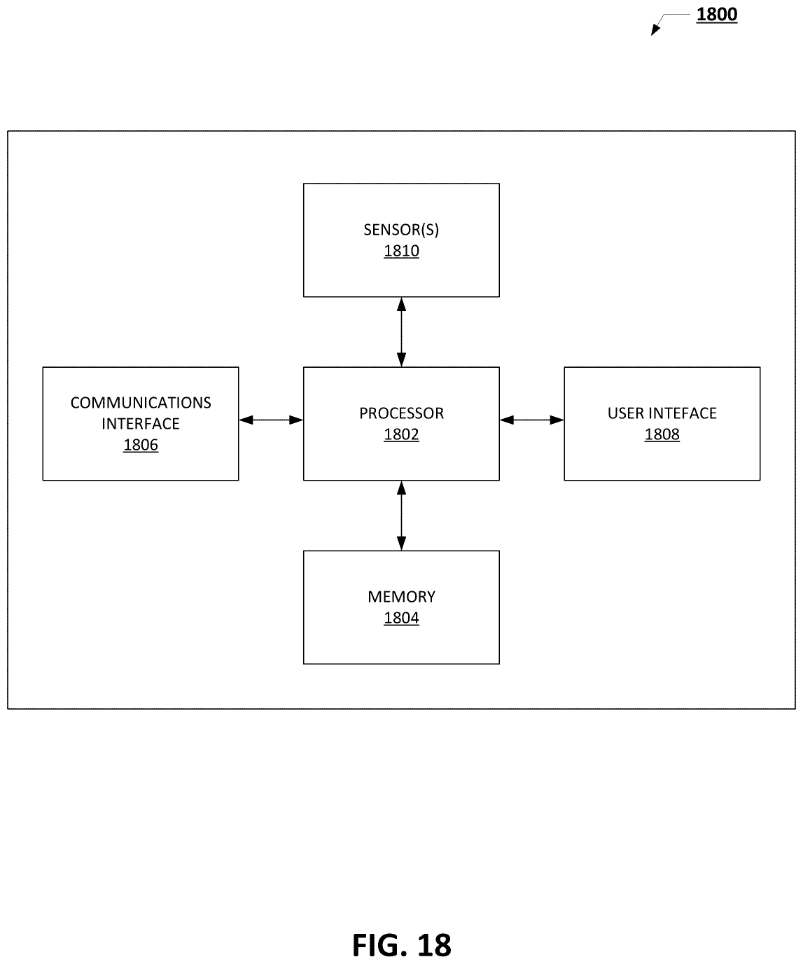

FIG. 18 illustrates a schematic diagram of an example embodiment of an apparatus 1800 that can be configured to execute, or cause execution of, one or more operations and/or methods described herein. For example, the apparatus 1800 embody an end device associated with an end user. Additionally, or alternatively, the apparatus 1800 may be embodied in a number of different ways such as, for example, as a PODAC module 106 and/or a respective MOPT 104a. The example apparatus 1800 includes or is otherwise in communication with a processor 1802, a memory 1804, a communications interface 1806 and a user interface 1808. As such, in some embodiments, although devices or elements are shown as being in communication with each other, hereinafter such devices or elements should be considered to be capable of being embodied within the same device or element and thus, devices or elements shown in communication should be understood to alternatively be portions of the same device or element.

In some embodiments, the processor 1802 (and/or co-processors or any other processing circuitry assisting or otherwise associated with the processor) may be in communication with the memory 1804 via a bus for passing information among components of the apparatus 1800. The memory 1804 may include, for example, one or more volatile and/or non-volatile memories. In other words, for example, the memory 1804 may be an electronic storage device (e.g., a computer readable storage medium) comprising gates configured to store data (e.g., bits) that may be retrievable by a machine (e.g., a computing device like the processor). The memory 1804 may be configured to store information, data, content, applications, instructions, or the like for enabling the apparatus 1800 to carry out various functions in accordance with an example embodiment of the present disclosure. For example, the memory 1804 could be configured to buffer input data for processing by the processor 1802. Additionally, or alternatively, the memory 1804 could be configured to store instructions for execution by the processor 1802.

The processor 1802 may be embodied in a number of different ways. For example, the processor 1802 may be embodied as one or more of various hardware processing means such as a coprocessor, a microprocessor, a controller, a digital signal processor (DSP), a processing element with or without an accompanying DSP, or various other processing circuitry including integrated circuits such as, for example, an ASIC (application specific integrated circuit), an FPGA (field programmable gate array), a microcontroller unit (MCU), a hardware accelerator, a special-purpose computer chip, or the like. As such, in some embodiments, the processor may include one or more processing cores configured to perform independently. A multi-core processor may enable multiprocessing within a single physical package. Additionally, or alternatively, the processor 1802 may include one or more processors configured in tandem via the bus to enable independent execution of instructions, pipelining and/or multithreading. The processor may be embodied as a microcontroller having custom bootloader protection for the firmware from malicious modification in addition to allowing for potential firmware updates.

In an example embodiment, the processor 1802 may be configured to execute instructions stored in the memory 1804 or otherwise accessible to the processor 1802. Alternatively, or additionally, the processor 1802 may be configured to execute hard coded functionality. As such, whether configured by hardware or software methods, or by a combination thereof, the processor 1802 may represent an entity (e.g., physically embodied in circuitry) capable of performing operations according to an embodiment of the present disclosure while configured accordingly. Thus, for example, when the processor 1802 is embodied as an ASIC, FPGA or the like, the processor 1802 may be specifically configured hardware for conducting the operations described herein. Alternatively, as another example, when the processor 1802 is embodied as an executor of software instructions, the instructions may specifically configure the processor 1802 to perform the algorithms and/or operations described herein when the instructions are executed. However, in some cases, the processor 1802 may be a processor of a specific device (e.g., the PODAC module 106) configured to employ an embodiment of the present disclosure by further configuration of the processor 1802 by instructions for performing the algorithms and/or operations described herein. The processor 1802 may include, among other things, a clock, an arithmetic logic unit (ALU) and logic gates configured to support operation of the processor 1802. In one embodiment, the processor 1802 may also include user interface circuitry configured to control at least some functions of one or more elements of the user interface 1808.

Meanwhile, the communications interface 1806 may include various components, such as a device or circuitry embodied in either hardware or a combination of hardware and software that is configured to receive and/or transmit data from the apparatus 1800 to a network, a server, or a particular user device operating the software application, for example. In this regard, the communications interface 1806 may include, for example, an antenna (or multiple antennas) and supporting hardware and/or software for enabling communications wirelessly. Additionally, or alternatively, the communications interface 1806 may include the circuitry for interacting with the antenna(s) to cause transmission of signals via the antenna(s) or to handle receipt of signals received via the antenna(s). For example, the communications interface 1806 may be configured to communicate wirelessly with a head-mounted display, such as via Wi-Fi (e.g., vehicular Wi-Fi standard 802.11p), Bluetooth, mobile communications standards (e.g., 3G, 4G, or 5G) or other wireless communications techniques. In some instances, the communications interface 1806 may alternatively or also support wired communication, which may communicate with a separate transmitting device (not shown). As such, for example, the communications interface 1806 may include a communication modem and/or other hardware/software for supporting communication via cable, digital subscriber line (DSL), universal serial bus (USB) or other mechanisms. For example, the communications interface 1806 may be configured to communicate via wired communication with other components of a computing device.

The user interface 1808 may be in communication with the processor 1802, such as the user interface circuitry, to receive an indication of a user input and/or to provide an audible, visual, mechanical, or other output to a user. As such, the user interface 1808 may include, for example, one or more buttons, light-emitting diodes (LEDs), a display, a speaker, and/or other input/output mechanisms. The user interface 1808 may also be in communication with the memory 1804 and/or the communications interface 1806, such as via a bus.

The communications interface 1806 may facilitate communication between the apparatus 1800 and various other devices, networks, or servers. The communications interface 1806 may be capable of operating in accordance with various first generation (1G), second generation (2G), 2.5G, third generation (3G) communication protocols, fourth generation (4G) communication protocols, fifth-generation (5G) communication protocols, Internet Protocol Multimedia Subsystem (IMS) communication protocols (e.g., session initiation protocol (SIP)), and/or the like. For example, a mobile terminal may be capable of operating in accordance with 2G wireless communication protocols IS-136 (Time Division Multiple Access (TDMA)), Global System for Mobile communications (GSM), IS-95 (Code Division Multiple Access (CDMA)), and/or the like. Also, for example, the mobile terminal may be capable of operating in accordance with 2.5G wireless communication protocols General Packet Radio Service (GPRS), Enhanced Data GSM Environment (EDGE), and/or the like. Further, for example, the mobile terminal may be capable of operating in accordance with 3G wireless communication protocols such as Universal Mobile Telecommunications System (UMTS), Code Division Multiple Access 2000 (CDMA2000), Wideband Code Division Multiple Access (WCDMA), Time Division-Synchronous Code Division Multiple Access (TD-SCDMA), and/or the like.

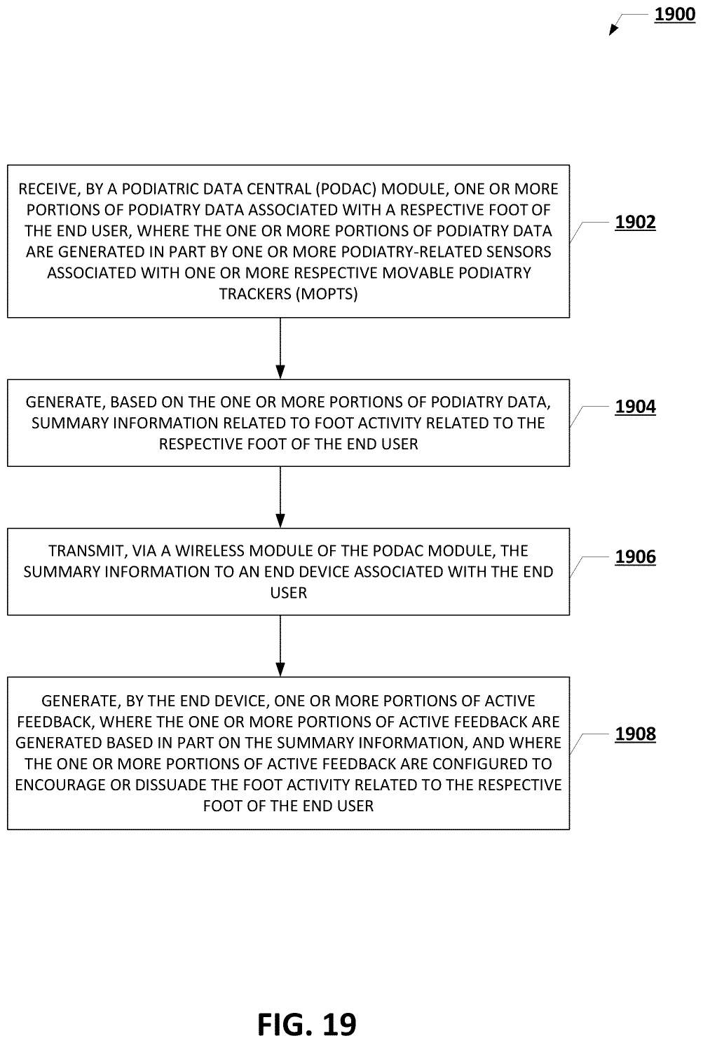

FIG. 19 illustrates a flowchart diagram for monitoring the condition and performance of the feet of a particular end user according to an example embodiment of the present disclosure. Specifically, FIG. 19 details a process 1900 related to various operations described herein. The process 1900 can be executed by, for example, the apparatus 1800. For example, at operation 1902, the apparatus 1800 comprises the means such as, for example, the processor 1802, the memory 1804, the communications interface 1806, the user interface 1808, the sensor(s) 1801, and/or a combination thereof configured to receive, by a podiatric data central (PODAC) module 106, one or more portions of podiatry data associated with a respective foot 112 of the end user, where the one or more portions of podiatry data are generated at least in part by one or more podiatry-related sensors 206a-n associated with one or more respective movable podiatry trackers (MOPTs) 104a-n.

At operation 1904, the apparatus 1800 comprises the means such as, for example, the processor 1802, the memory 1804, the communications interface 1806, the user interface 1808, the sensor(s) 1801, and/or a combination thereof configured to generate, based at least in part on the one or more portions of podiatry data, summary data related to foot activity related to the respective foot 112 of the end user.

At operation 1906, the apparatus 1800 comprises the means such as, for example, the processor 1802, the memory 1804, the communications interface 1806, the user interface 1808, the sensor(s) 1801, and/or a combination thereof configured to transmit, via a wireless module 208 of the PODAC module 106, the summary data to an end device 108 associated with the end user.

At operation 1908, the apparatus 1800 comprises the means such as, for example, the processor 1802, the memory 1804, the communications interface 1806, the user interface 1808, the sensor(s) 1801, and/or a combination thereof configured to generate, by the end device 108, one or more portions of active feedback, where the one or more portions of active feedback are generated based at least in part on the summary data, and where the one or more portions of active feedback are configured to encourage or discourage the foot activity related to the respective foot 112 of the end user.

1. A computer-implemented comprising:
receiving, by one or more processors, podiatry data associated with a foot of an end user, wherein the podiatry data is generated by one or more podiatry-related sensors;
generating, by the one or more processors and based at least in part on the podiatry data, summary data related to foot activity related to the respective foot of the end user;
providing, by the one or more processors, the summary data for an end device associated with the end user, wherein the end devices is configured to generate active feedback based at least in part on the summary data, wherein active feedback is configured to encourage or discourage a particular foot activity related to the foot of the end user.

2. The computer-implemented method of claim 1, wherein one or more respective movable podiatry trackers (MOPTs) comprise an active feedback unit and a sensor processing unit.

3. The computer-implemented method of claim 2, wherein the sensor processing unit is configured to collect raw data from the one or more podiatry-related sensors.

4. The computer-implemented method of claim 2, wherein the active feedback unit is configured to provide one or more types of physical feedback, wherein the one or more types of physical feedback comprise at least one of vibration, heat, or sound.

5. The computer-implemented method of claim 4, wherein at least one of the one or more portions of the active feedback or the one or more types of the physical feedback are determined at least in part by a recommendation system associated with the end device.

6. The computer-implemented method of claim 6 further comprising generating, based at least in part on one or more portions of data comprised in the information database, a heatmap associated with the respective foot of the end user.

7. The computer-implemented method of claim 5, wherein the end device is configured to generate one or more predictions related to at least one of a current foot activity or a future foot activity.

8. The computer-implemented method of claim 5, wherein the end device is configured to provide one or more recommendations based at least on the one or more predictions, wherein the one or more recommendations comprise at least one of a recommendation to sustain a current foot activity, a recommendation to cease a current foot activity, a recommendation to adjust a stance, or a recommendation to adjust a gait.

9. The computer-implemented method of claim 1 further comprising:
determining, based at least in part on image data captured by a camera associated with the end device, one or more locations of the one or more respective MOPTs relative to the respective foot of the end user.

10. The computer-implemented method of claim 1, wherein a podiatric data central (PODAC) module and one or more respective movable podiatry trackers (MOPTs) comprise are characterized by a multi-layered construction, wherein the multi-layered construction comprises at least one of one or more of electronic-safe epoxy, one or more surface mount components, one or more flexible printed circuit board (PCB) substrates, or one or more adhesives.

11. The computer-implemented method of claim 10, wherein the PODAC module or the one or more MOPTs are configured to be embedded in existing footwear.

12. The computer-implemented method of claim 10, wherein the one or more MOPTs are constructed to feature a plurality of functional extensions of various lengths, wherein the plurality of functional extensions of various lengths comprise one or more podiatry-related sensors.

13. A system comprising memory and one or more processors communicatively coupled to the memory, the one or more processors configured to:
receive podiatry data associated with a foot of an end user, wherein the podiatry data is generated by one or more podiatry-related sensors;
generate, based at least in part on the podiatry data, summary data related to foot activity related to the respective foot of the end user;
providing, by the one or more processors, the summary data for an end device associated with the end user, wherein the end devices is configured to generate active feedback based at least in part on the summary data, wherein active feedback is configured to encourage or discourage a particular foot activity related to the foot of the end user.

14. The system of claim 13, wherein one or more respective movable podiatry trackers (MOPTs) comprise an active feedback unit and a sensor processing unit.

15. The system of claim 14, wherein the sensor processing unit is configured to collect raw data from the one or more podiatry-related sensors.

16. The system of claim 14, wherein the active feedback unit is configured to provide one or more types of physical feedback, wherein the one or more types of physical feedback comprise at least one of vibration, heat, or sound.

17. The system of claim 16, wherein a podiatric data central (PODAC) module and one or more respective movable podiatry trackers (MOPTs) comprise are characterized by a multi-layered construction, wherein the multi-layered construction comprises at least one of one or more of electronic-safe epoxy, one or more surface mount components, one or more flexible printed circuit board (PCB) substrates, or one or more adhesives.

18. One or more non-transitory computer-readable storage media including instructions that, when executed by one or more processors, cause the one or more processors to:
receive podiatry data associated with a foot of an end user, wherein the podiatry data is generated by one or more podiatry-related sensors;
generate, based at least in part on the podiatry data, summary data related to foot activity related to the respective foot of the end user;
providing, by the one or more processors, the summary data for an end device associated with the end user, wherein the end devices is configured to generate active feedback based at least in part on the summary data, wherein active feedback is configured to encourage or discourage a particular foot activity related to the foot of the end user.

19. The one or more non-transitory computer-readable storage media of claim 18, wherein one or more respective movable podiatry trackers (MOPTs) comprise an active feedback unit and a sensor processing unit.

20. The one or more non-transitory computer-readable storage media of claim 19, wherein the sensor processing unit is configured to collect raw data from the one or more podiatry-related sensors.
